(19) United States
(12) Patent Application Publication
(10) Pub. No.: US 2023/0029483 A1
(43) Pub. Date: Feb. 2, 2023

(54) FABRICATION SUPPORT APPARATUS OF LAYERED CELL SHEET, FABRICATION SUPPORT SYSTEM, FABRICATION SUPPORT METHOD, AND COMPUTER-READABLE MEDIUM

(71) Applicant: EVIDENT CORPORATION, Nagano (JP)

(72) Inventors: Elena SHIMOJI, Tokyo (JP); Yoko KAMATO, Tokyo (JP); Takuma DEZAWA, Tokyo (JP)

(73) Assignee: EVIDENT CORPORATION, Nagano (JP)

(21) Appl. No.: 17/687,814

(22) Filed: Mar. 7, 2022

(30) Foreign Application Priority Data
Jul. 27, 2021 (JP) 2021-122277

(51) Int. Cl. G05B 19/402; G06T 7/00; G06T 7/60; G06T 7/70; G06T 7/11

(52) U.S. Cl. CPC G05B 19/402; G06T 7/0006; G06T 7/60; G06T 7/70; G06T 7/11; G06T 2207/30108; G05B 2219/32287

(57) ABSTRACT

A fabrication support apparatus of a layered cell sheet in which a plurality of cell sheets are layered on one another includes circuitry. The circuitry is configured to acquire optical information that is information regarding optical properties of the layered cell sheet; to calculate a thickness distribution of the layered cell sheet based on the acquired optical information; to determine a layering position of a cell sheet to be newly layered on the layered cell sheet based on the calculated thickness distribution; and to output information of the determined layering position.

### CROSS REFERENCE TO RELATED APPLICATIONS

This application is based upon and claims the benefit of priority from prior Japanese Patent Application No. 2021-122277, filed Jul. 27, 2021, the entire contents of which are incorporated herein by this reference.

### FIELD

The disclosure of the present specification relates to a fabrication support apparatus of a layered cell sheet, a fabrication support system, a fabrication support method, and a computer-readable medium.

### BACKGROUND

In the field of regenerative medicine, a cell sheet obtained by culturing, into a sheet shape, cells collected from a living body has attracted attention. The cell sheet aims for regeneration and functional recovery of a damaged tissue by being pasted thereonto.

The cell sheet is coupled to a biological tissue by an adhesion protein, and accordingly, pasting thereof to a damaged portion of the biological tissue just enables transplantation thereof in a short time without sewing. Further, it is known that use of the cell sheet reduces an outflow of cells, which is a problem in therapy using injection of a cell suspension, and also increases an effect of the therapy. Therefore, at present, clinical applications are being started in a variety of fields.

Further, a lot of proposals have also been made about a layering technology for layering cell sheets in order to transplant a larger number of cells at one time. For example, JP 2012-147687 A describes a technology for determining the number of layered cell sheets.

### SUMMARY

A fabrication support apparatus according to an aspect of the present invention is a fabrication support apparatus of a layered cell sheet in which a plurality of cell sheets are layered on one another. the apparatus comprising circuitry. The circuitry is configured: to acquire optical information that is information regarding optical properties of the layered cell sheet; to calculate a thickness distribution of the layered cell sheet based on the acquired optical information; to determine a layering position of a cell sheet to be newly layered on the layered cell sheet based on the calculated thickness distribution; and to output information of the determined layering position.

A fabrication support system according to an aspect of the present invention includes: the fabrication support apparatus according to the above-described aspect; and a layering device that layers a new cell sheet on the layered cell sheet based on the information of the layering position, the information being output by the fabrication support apparatus.

A fabrication support method according to an aspect of the present invention is a fabrication support method of a layered cell sheet in which a plurality of cell sheets are layered on one another, the method including: acquiring optical information that is information regarding optical properties of the layered cell sheet; calculating a thickness distribution of the layered cell sheet based on the acquired optical information; determining a layering position of a cell sheet to be newly layered on the layered cell sheet based on the calculated thickness distribution; and outputting information of the layering position determined by the determination unit.

A non-transitory computer-readable medium according to an aspect of the present invention is a non-transitory computer-readable medium that stores a program for causing a computer of a fabrication support apparatus of a layered cell sheet in which a plurality of cell sheets are layered on one another to execute processing: for acquiring optical information that is information regarding optical properties of the layered cell sheet; for calculating a thickness distribution of the layered cell sheet based on the acquired optical information; for determining a layering position of a cell sheet to be newly layered on the layered cell sheet based on the calculated thickness distribution; and for outputting information of the layering position determined by the determination unit.

### BRIEF DESCRIPTION OF THE DRAWINGS

The present invention will be more apparent from the following detailed description when the accompanying drawings are referenced.

FIG. 1 is a diagram illustrating a configuration of a system according to a first embodiment;

FIG. 2 is a diagram illustrating a functional configuration of a control device according to the first embodiment;

FIG. 3 is an example of a flowchart of processing performed by the system according to the first embodiment;

FIG. 4 is an example of a flowchart of layering support processing according to the first embodiment;

FIG. 5 is a flowchart of determination processing according to the first embodiment;

FIG. 6 is a view illustrating region values of partial regions;

FIG. 7 is a flowchart of target partial region selection processing according to the first embodiment;

FIG. 8 is a view illustrating an example of a layering position determined by the determination processing according to the first embodiment;

FIG. 9 is a view illustrating another example of the layering position determined by the determination processing according to the first embodiment;

FIG. 10 is an example of a flowchart of processing performed by a system according to a second embodiment;

FIG. 11 is a view illustrating an example of a screen on which a layering position is displayed;

FIG. 12 is a diagram illustrating a functional configuration of a control device according to a third embodiment;

FIG. 13 is an example of a flowchart of layering support processing according to the third embodiment;

FIG. 14 is a view illustrating an example of disposition of cell sheets stopped being layered;

FIG. 15 is an example of a flowchart of layering support processing according to a fourth embodiment;

FIG. 16 is a view illustrating an example of a screen to propose the stop of the layering;

FIG. 17 is a diagram illustrating a functional configuration of a control device according to a fifth embodiment;

FIG. 18 is an example of a flowchart of layering support processing according to a fifth embodiment;

FIG. 19 is a view illustrating an example of a layering position determined by determination processing according to the fifth embodiment;

FIG. 20 is an example of a flowchart of layering support processing according to a sixth embodiment;

FIG. 21 is a view illustrating another example of the screen to propose the stop of the layering;

FIG. 22 is a diagram illustrating a functional configuration of a control device according to a seventh embodiment;

FIG. 23 is an example of a flowchart of layering support processing according to a seventh embodiment;

FIG. 24 is a view illustrating an example of a model image;

FIG. 25 is a diagram illustrating a configuration of a system according to an eighth embodiment;

FIG. 26 is a diagram illustrating a functional configuration of a control device according to the eighth embodiment;

FIG. 27 is a view illustrating a relationship between types of the cell sheets and light quantities detected therefrom;

FIG. 28 is a view for explaining a method of determining a layered state of different types of the cell sheets; and

FIG. 29 is a diagram illustrating a hardware configuration of a computer for achieving the control device.

### DETAILED DESCRIPTION

Incidentally, in order to efficiently fabricate the layered cell sheet, it is desirable to change disposition of a new cell sheet according to a layered situation of the cell sheet.

Considering such circumstances, embodiments of the present invention will be described hereinafter.

(First Embodiment)

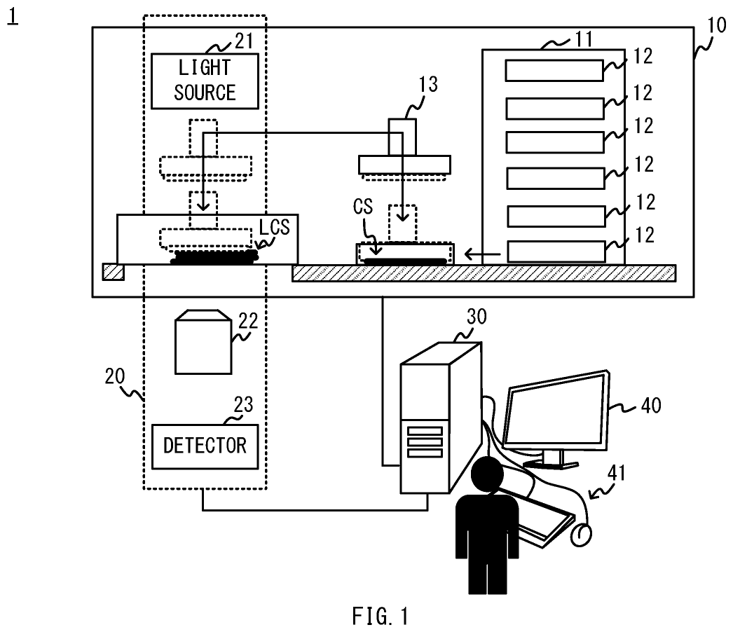

FIG. 1 is a diagram illustrating a configuration of a system 1 according to the present embodiment. Referring to FIG. 1, the system 1 will be described. The system 1 is a system that supports the fabrication of the layered cell sheet. For example, by instructing the system 1 on a size and a thickness which a user requests for the layered cell sheet (hereinafter, this size and the thickness will also be referred to as a target size and a target thickness, respectively), thereby the system 1 executes processing programmed in advance, and automatically fabricates a layered cell sheet that meets the request.

As illustrated in FIG. 1, the system 1 includes a layering device 10, a measuring device 20, and a control device 30. The system 1 may further include a display device 40 and an input device 41.

The layering device 10 layers cell sheets CS, and fabricates a layered cell sheet LCS. The layering device 10 includes: a mechanism that takes out culture vessels 12 from an incubator 11 and conveys the culture vessels 12 to a predetermined position; and a manipulator 13 that peels off the cell sheets CS from the culture vessels 12 and layers the cell sheets CS. Layering work by the layering device 10 is controlled by the control device 30. Though not particularly limited, the manipulator 13 may be such a stamp-type device, for example, as illustrated in FIG. 1, which thrusts each of the cell sheets CS against a supporting body, thereby transferring and conveying the cell sheet CS from the culture vessel 12 to the supporting body.

The measuring device 20 measures optical properties of the layered cell sheet LCS fabricated by the layering device 10, and generates information regarding the optical properties (hereinafter, this information will be referred to as optical information). The measuring device 20 includes: a light source 21 that emits light to be applied to the layered cell sheet LCS; an optical system 22 that collects light passing through the layered cell sheet LCS; and a detector 23 that detects the light collected by the optical system 22. The measuring device 20 may measure a transmittance to a specific wavelength or transmittances to a plurality of different wavelengths as the optical properties of the layered cell sheet LCS. Note that the measurement of the transmittance may mean generation of transmittance information by directly calculating the transmittance, or may mean generation of information that mirrors the transmittance. Hence, the optical information regarding the optical properties of the layered cell sheet LCS may be an image of the layered cell sheet LCS, which mirrors the transmittance. For example, the measuring device 20 is a microscope that acquires a bright-field image by transillumination.

The control device 30 is an example of the fabrication support apparatus of the layered cell sheet LCS. The control device 30 controls the layering device 10 on the basis of the optical information generated by the measuring device 20, thereby supporting the fabrication of the layered cell sheet LCS. More specifically, the control device 30 calculates a layering position that is a position of the cell sheet CS to be newly layered on the layered cell sheet LCS, and controls the layering device 10 by using information of the calculated layering position. Note that, hereinafter, the layering position refers to a position of the cell sheet CS to be newly layered on the layered cell sheet LCS, more specifically, refers to a position serving as a target when the cell sheet CS is newly layered on the layered cell sheet LCS. The layering position is, for example, a position of a region having a shape and a size which are equivalent to those of the new cell sheet CS. However, when the shape and size of the new cell sheet CS are not known, the layering position may be a position of a region having predetermined shape and size, or may be a position of a region having a shape and a size which are expected for the new cell sheet CS. Moreover, the layering position may be a position of one point on the layered cell sheet LCS. Moreover, the layering position may be an aggregate of positions of two or more points on the layered cell sheet LCS.

The display device 40 is an arbitrary display device, and for example, is a liquid crystal display, an organic EL display, or the like. On the display device 40, for example, a setting screen is displayed, which serves for inputting a variety of setting information such as a size and a thickness which the user requests for the layered cell sheet LCS. Moreover, an image of the layered cell sheet LCS, which is generated by the measuring device 20, or the like may be displayed on the display device 40.

The input device 41 is an arbitrary input device, and for example, is a keyboard, a mouse, a touch device and the like. By using the input device 41, the user performs input, change and the like of the variety of setting information.

In the system 1 configured as described above, the control device 30 calculates the layering position on the basis of the optical information generated by the measuring device 20, and controls the layering device 10 by using the information of the calculated layering position. Further, in the layering device 10, the manipulator 13 disposes the cell sheet CS at the layering position indicated by the control device 30, and layers the cell sheet CS thereon. Thus, the cell sheet CS will be disposed at an optimal position according to the layered situation of the cell sheet in the fabrication process of the layered cell sheet LCS. Therefore, in accordance with the system 1, such a layered cell sheet LCS with specifications (the thickness and the area) requested by the user can be fabricated efficiently. Further, the optical information for use in the event of calculating the layering position is generated by non-contact and non-invasive measurement by the measuring device 20. Therefore, avoided is application of an excessive damage to the layered cell sheet LCS for the purpose of calculating the layering position. Hence, in accordance with the system 1, fabrication efficiency of the layered cell sheet LCS can be improved without sacrificing quality thereof.

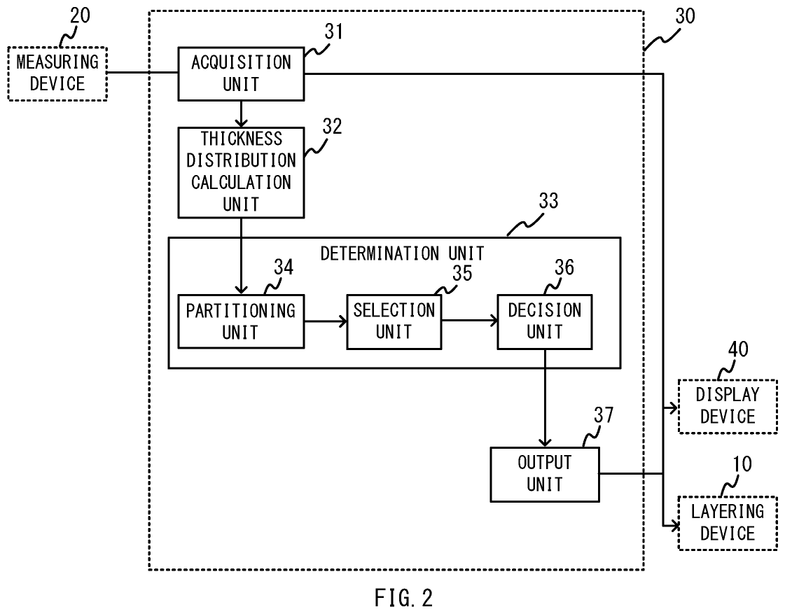

FIG. 2 is a diagram illustrating a functional configuration of the control device 30 according to the present embodiment. Referring to FIG. 2, a description will be given below of such a configuration related to a fabrication support function provided in the control device 30.

In order to support the fabrication of the layered cell sheet LCS by the system 1, the control device 30 includes an acquisition unit 31, a thickness distribution calculation unit 32, a determination unit 33, and an output unit 37 as illustrated in FIG. 2.

The acquisition unit 31 acquires the optical information that is the information regarding the optical properties of the layered cell sheet LCS. The acquisition unit 31 acquires the optical information generated by the measuring device 20. The optical information is, for example, the image of the layered cell sheet LCS.

The thickness distribution calculation unit 32 calculates a thickness distribution of the layered cell sheet LCS on the basis of the optical information acquired by the acquisition unit 31. For example, by using information of a transmittance per unit thickness of the cell sheet CS, the thickness distribution calculation unit 32 calculates the thickness distribution of the layered cell sheet LCS from the image of the layered cell sheet LCS, which is the optical information.

On the basis of the thickness distribution calculated by the thickness distribution calculation unit 32, the determination unit 33 determines the layering position of the cell sheet CS to be newly layered on the layered cell sheet LCS. For example, on the basis of the calculated thickness distribution, the determination unit 33 may decide the layering position so that the thickest region of the layered cell sheet LCS and the cell sheet to be newly layered overlap each other. Moreover, the determination unit 33 may decide the layering position according to criteria different depending on whether or not the layered cell sheet LCS has a region that has reached the target thickness. When the layered cell sheet LCS has no region that has reached the target thickness, the determination unit 33 may decide the layering position on the basis of the thickness distribution so that the thickest region of the layered cell sheet LCS and the cell sheet CS to be newly layered overlap each other. Meanwhile, when the layered cell sheet LCS has the region that has reached the target thickness, the determination unit 33 may decide the layering position on the basis of the thickness distribution so that a region that has not reached the target thickness and is adjacent to the region that has reached the target thickness and the cell sheet CS to be newly layered overlap each other. Desirably, as illustrated in FIG. 2, the determination unit 33 includes a partitioning unit 34, a selection unit 35, and a decision unit 36.

The partitioning unit 34 partitions the region of the layered cell sheet LCS into one or more partial regions on the basis of the thickness distribution calculated by the thickness distribution calculation unit 32. A partitioning method is not particularly limited; however, for example, the region of the layered cell sheet LCS may be partitioned as follows.

First, the partitioning unit 34 classifies the respective pixels of the image of the layered cell sheet LCS according to thicknesses (that is, pixel values) thereof at positions of the pixels. The pixels may be classified not only for each pixel but also for each set of a predetermined number of pixels (hereinafter, referred to as “pixel set”). In that case, a thickness that represents each pixel set may be decided on the basis of a mean value or median value of the values of the predetermined number of pixels, and the pixels may be classified on the basis of the decided thickness.

Note that, desirably, the partitioning unit 34 classifies the pixels (pixel sets) at least into a class corresponding to a thickness range of the target thickness or more and a class corresponding to a thickness range of less than the target thickness. Desirably, the thickness range corresponding to each of the classes includes, for example, a thickness approximately equivalent to one cell sheet CS. Thus, the respective pixels can be substantially classified according to the layered number of cell sheets CS. However, the thickness ranges corresponding to the respective classes may be the same, or may be different.

When the pixels (the pixel sets) are classified, the partitioning unit 34 classifies the respective continuous regions, which are classified into the same classes, into the partial regions. For example, by such a method as described above, the region of the layered cell sheet LCS can be partitioned into one or more partial regions.

The selection unit 35 selects a target partial region taken as a layering target from among the one or more partial regions on the basis of at least one of sizes and thicknesses of the one or more partial regions defined by the partitioning unit 34. Herein, the layering target means a target on which the cell sheet CS that is new is to be layered.

The size of each of the partial regions is, for example, an area, and may be obtained by conversion using the number of pixels (the number of pixel sets) which belong to the partial region. The thickness of each partial region may be a thickness that represents the class corresponding to the partial region. For example, the thickness is a median value of the thickness range corresponding to the class, a maximum value thereof, a minimum value thereof, or the like. Further, the thickness of each partial region may be calculated on the basis of the thickness of the layered cell sheet LCS at each of positions in the partial region, and for example, may be a mean value of pixel values of the pixels belonging to the partial region, a mean value thereof, a maximum value thereof, a minimum value thereof, or the like.

Desirably, the selection unit 35 selects the target partial region, for example, on the basis of the target size and target thickness of the layered cell sheet LCS in addition to the size and thickness of the partial region. By using these pieces of information, it is possible to decide a layering position efficient in terms of fabricating the layered cell sheet LCS having the target size and the target thickness.

The decision unit 36 decides the layering position so that the target partial region selected by the selection unit 35 and the cell sheet CS to be newly layered overlap each other. Specifically, the decision unit 36 may decide the layering position, for example, so that an overlapping region of the target partial region and the newly layered cell sheet CS is maximized Further, the decision unit 36 may decide the layering position, for example, so that the center of the cell sheet CS to be newly layered is disposed so as to be in the center of the target partial region.

The output unit 37 outputs information of the layering position determined by the decision unit 36. For example, the output unit 37 outputs the information of the layering position to the layering device 10. Thus, the layering device 10 layers the cell sheet CS on the basis of the information of the layering position, which is output from the output unit 37.

Note that the output unit 37 may output the information of the layering position to other devices, for example, to the display device 40. Further, the output unit 37 may output the information of the layering position in combination with other information. For example, the output unit 37 may output the information of the layering position in combination with the image generated by the measuring device 20, or may output a synthetic image in which a mark indicating the layering position is superposed on the image.

In the control device 30 configured as described above, the thickness distribution of the layered cell sheet LCS is calculated on the basis of the optical properties measured by the measuring device 20, the layering position that is appropriate is determined from the region of the layered cell sheet LCS on the basis of the thickness distribution, and the information of the layering position is output to the other devices of the system 1. Thus, the system 1 can fabricate the layered cell sheet LCS efficiently by using the information of the layering position, which is output by the control device 30. Hence, in accordance with the control device 30, the fabrication efficiency of the layered cell sheet can be improved.

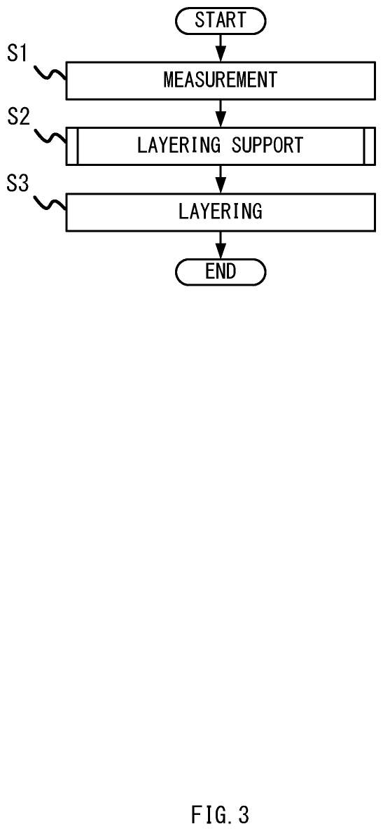

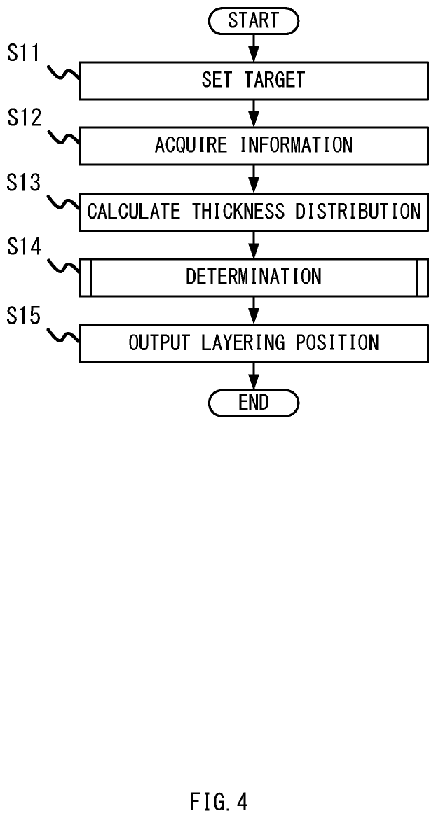

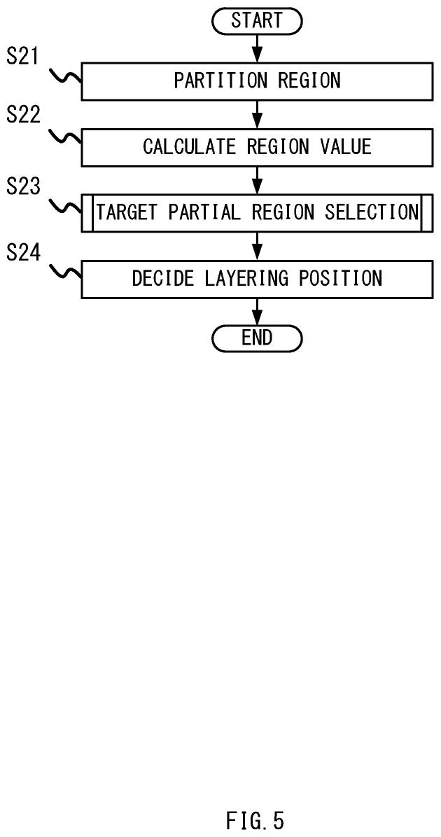

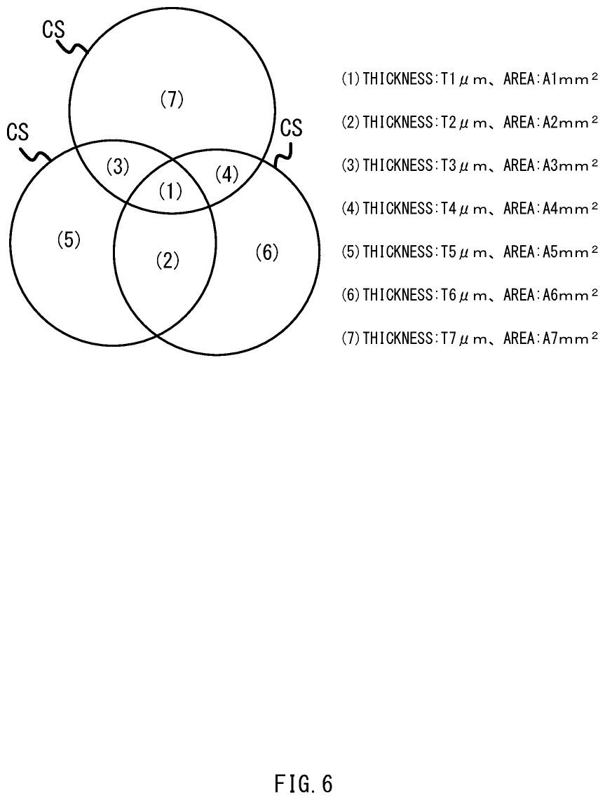

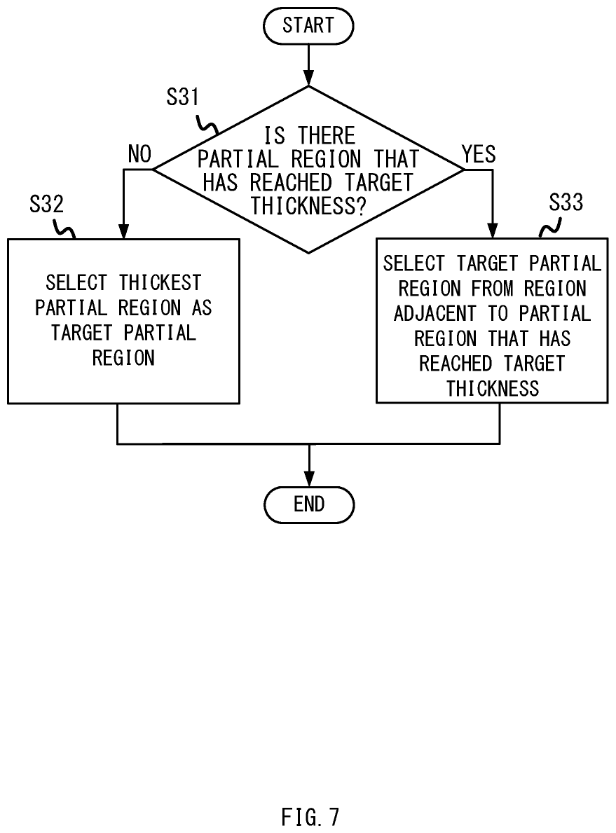

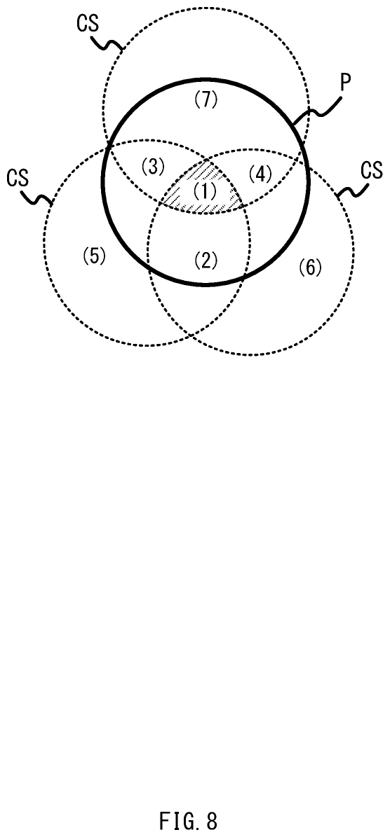

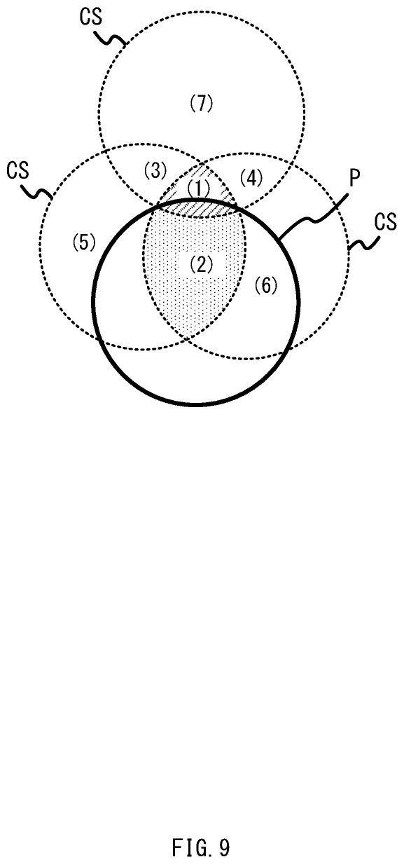

FIG. 3 is an example of a flowchart of the processing performed by the system 1 according to the present embodiment. FIG. 4 is an example of a flowchart of layering support processing according to the present embodiment. FIG. 5 is a flowchart of determination processing according to the present embodiment. FIG. 6 is a view illustrating region values of partial regions. FIG. 7 is a flowchart of target partial region selection processing according to the present embodiment. FIGS. 8 and 9 are views each illustrating an example of a layering position decided by the determination processing according to the present embodiment. Referring to FIGS. 3 to 9, a specific description will be given below of the processing performed by the system 1 according to the present embodiment.

For example, automatic fabrication processing of the layered cell sheet LCS, which is illustrated in FIG. 3, is started in such a manner that, by using the input device 41, the user instructs the control device 30 to automatically fabricate the layered cell sheet LCS. The automatic fabrication processing illustrated in FIG. 3 includes: measurement processing (step S1) performed by the measuring device 20; layering support processing (step S2) performed by the control device 30; and layering processing (step S3) performed by the layering device 10. Note that these pieces of processing are repeatedly performed, for example, until the layered cell sheet LCS having the target size and the target thickness is completed.

First, in the measurement processing of step S1, the measuring device 20 images the layered cell sheet LCS in which a plurality of the cell sheets CS are layered on one another, and outputs the image thus generated to the control device 30. For example, the control device 30 that has received the image executes a predetermined program, and starts layering support processing illustrated in FIG. 4.

First, the control device 30 sets targets for the layered cell sheet LCS to be fabricated (step S11). Herein, the control device 30 sets, for example, the target size and target thickness of the layered cell sheet LCS. The control device 30 may set the target size and the target thickness by acquiring the information input by the user, or may set the target size and the target thickness by reading out a setting file having the same described therein.

When the targets are set, in the control device 30, the acquisition unit 31 acquires information received from the measuring device 20, that is, the image in this example (step S12), and the thickness distribution calculation unit 32 calculates the thickness distribution of the layered cell sheet LCS, which is in the image, on the basis of the image (step S13).

Thereafter, the determination unit 33 performs determination processing, which is illustrated in FIG. 5, by using the thickness distribution calculated in step S13 (step S14). In the determination processing, first, the partitioning unit 34 partitions the region of the layered cell sheet LCS into the partial regions (step S21). Next, the selection unit 35 calculates the region values of the respective partial regions (step S22). Herein, for example, as illustrated in FIG. 6, the selection unit 35 calculates, as the region values, the area and thickness of the partial region for each of the partial regions. FIG. 6 illustrates an example in which the layered cell sheet LCS is partitioned into seven partial regions, each of which is subjected to calculation of the thickness and the area. Note that the thickness of each partial region is a thickness that represents the partial region, and for example, is a mean thickness of the partial region.

When the region values are calculated, the selection unit 35 performs target region selection processing illustrated in FIG. 7 (step S23). Herein, first, the selection unit 35 determines whether or not there is a partial region that has reached the target thickness by comparing the region value (thickness) calculated in step S22 with the target thickness (step S31).

When there is no partial region that has reached the target thickness, the selection unit 35 decides, as the target partial region, the thickest partial region from among the one or more partial regions (step S32). For example, when the thickest partial region (1) has not reached the target thickness, then as illustrated in FIG. 8, the selection unit 35 decides the partial region (1) as the target partial region.

Meanwhile, when there is a partial region that has reached the target thickness, the selection unit 35 decides, as the target partial region, the largest partial region from among such partial regions which have not reached the target thickness and are adjacent to the partial region that has reached the target thickness (step S33). For example, when the thickest partial region (1) has reached the target thickness, then as illustrated in FIG. 9, the selection unit 35 decides, as the target partial region, a partial region (2) with the largest area from among such partial regions (2) to (4) adjacent to the partial region (1).

When the target partial region is decided, the decision unit 36 decides the layering position (step S24). For example, when the partial region (1) is selected as the target partial region, then as illustrated in FIG. 8, the decision unit 36 decides the layering position P so that the partial region (1) and the layering position P overlap each other. Further, when the partial region (2) is selected as the target partial region, then as illustrated in FIG. 9, the decision unit 36 decides the layering position P so that the partial region (2) and the layering position P overlap each other.

When the layering position is decided, the output unit 37 outputs, to the layering device 10, information of the layering position decided in step S24 (step S15), and the control device 30 ends the layering support processing illustrated in FIG. 4.

Thereafter, in step S3, the layering device 10 disposes the new cell sheet CS at the layering position on the layered cell sheet LCS on the basis of the information of the layering position, which is received from the control device 30, and thereby layers the cell sheet CS.

The system 1 repeats the processing of FIG. 3, whereby the layered cell sheet LCS having the target size and the target thickness can be fabricated efficiently. Particularly, in the system 1, in a state in which the layered cell sheet LCS has not reached the target thickness, the control device 30 decides the layering position so that the cell sheet CS overlaps the thickest portion. Thus, it is possible to reduce a wasted region in the fabrication process of the layered cell sheet, and as a result, the layered cell sheet LCS can be fabricated efficiently. Further, when at least a part of the layered cell sheet LCS has reached the target thickness, the control device 30 decides the layering position of the cell sheet CS so that the cell sheet CS overlaps a region adjacent to that region as the at least a part. Thus, the cell sheet CS will be disposed so as to increase the area of the layered cell sheet LCS having the target thickness, and accordingly, such a layered cell sheet LCS that has a required area can be fabricated efficiently. Further, the largest region is selected from among the adjacent regions to decide the layering position, whereby such a layered cell sheet LCS that varies less in thickness can be fabricated. As described above, in accordance with the control device 30 according to the present embodiment, the fabrication efficiency of the layered cell sheet can be improved.

Further, the control device 30 roughly decides the region, on which the cell sheet CS is to be layered, on the basis of the region value calculated for each of the partial regions. That is, the control device 30 makes a decision per unit region, and decides the layering position within such a loose restriction. The layering position is decided in such a procedure, whereby it is possible to decrease sensitivity to some positional shift that occurs when the cell sheet CS is actually layered. Therefore, the layered cell sheet can be fabricated at stable fabrication efficiency without excessively depending on positioning accuracy of the layering device 10. As described above, in accordance with the control device 30 according to the present embodiment, the stability of the fabrication efficiency of the layered cell sheet can also be improved.

(Second Embodiment)

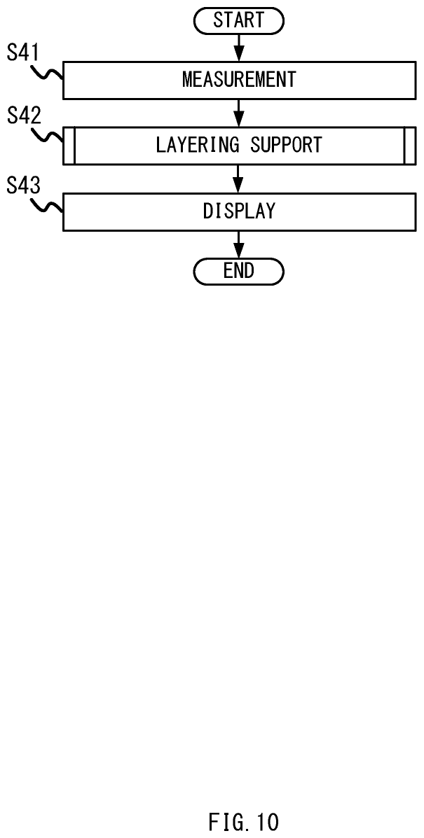

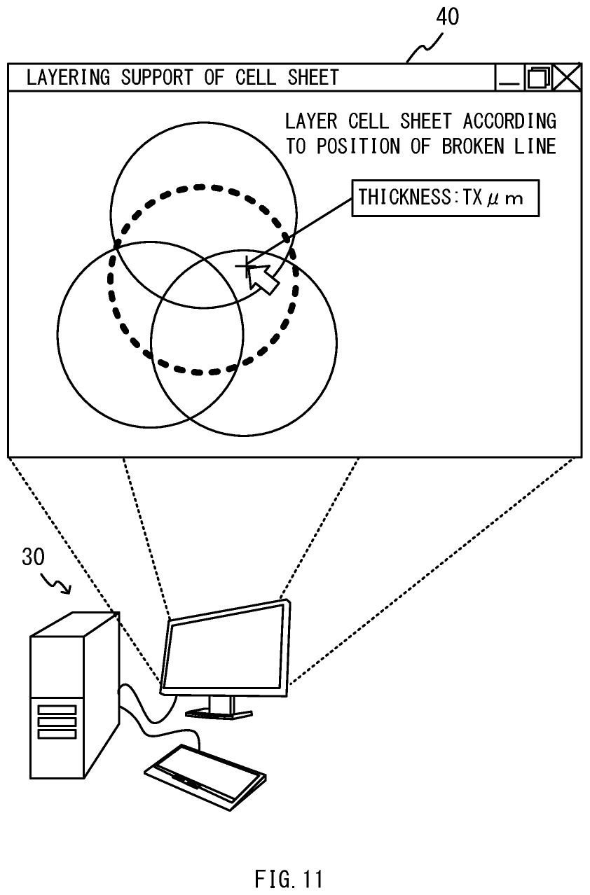

FIG. 10 is an example of a flowchart of processing performed by a system according to the present embodiment. FIG. 11 is a view illustrating an example of a screen on which the layering position is displayed. Referring to FIGS. 10 and 11, a description will be given below of the present embodiment.

Like the system 1, the system according to the present embodiment is a system that supports the fabrication of the layered cell sheet. The system according to the present embodiment is different from the system 1 in that the layering work of the cell sheet CS is manually performed. For example, the manipulator 13 of the layering device 10 is manually operated. Moreover, a control device included in the system is different from the control device 30 in outputting, to the display device 40, a synthetic image including the layering position. Note that the present embodiment is also applicable to a case where the user layers the cell sheet CS by using an instrument such as tweezers without using the layering device 10. That is, in the system according to the present embodiment, the layering device 10 may be omitted.

Manual fabrication support processing for the layered cell sheet LCS, which is illustrated in FIG. 10, includes: measurement processing (step S41) performed by the measuring device 20; layering support processing (step S42) performed by the control device 30; and display processing (step S43) performed by the display device 40. Note that the pieces of processing in steps S41 and S42 are similar to the pieces of processing in steps S1 and S2 in FIG. 3.

In step S43, the display device 40 displays information of the layering position, which is output by the control device that is a fabrication support apparatus. Specifically, for example, as illustrated in FIG. 11, the display device 40 displays the information of the layering position on the image of the layered cell sheet LCS in a superimposing manner. Thus, while viewing the synthetic image displayed on the display device 40, the user can dispose the new cell sheet CS on the layered cell sheet LCS by operating the manipulator 13. Further, the user may dispose the new cell sheet CS on the layered cell sheet LCS by the instrument such as tweezers. As described above, in accordance with the system and the control device according to the present embodiment, the layering work of the cell sheet CS, which is manually performed by the user, can be supported. Hence, also in accordance with the present embodiment, the fabrication efficiency of the layered cell sheet can be improved.

Note that the control device 30 may display variety of information on the display device 40 according to an operation by the user. For example, a cursor is placed on an arbitrary position of the image of the layered cell sheet LCS displayed on the display device 40, whereby a thickness of the layered cell sheet LCS at that position may be displayed, for example, as illustrated in FIG. 11. Since the thickness distribution of the layered cell sheet LCS is calculated in advance, such display as described above can be performed with ease.

(Third Embodiment)

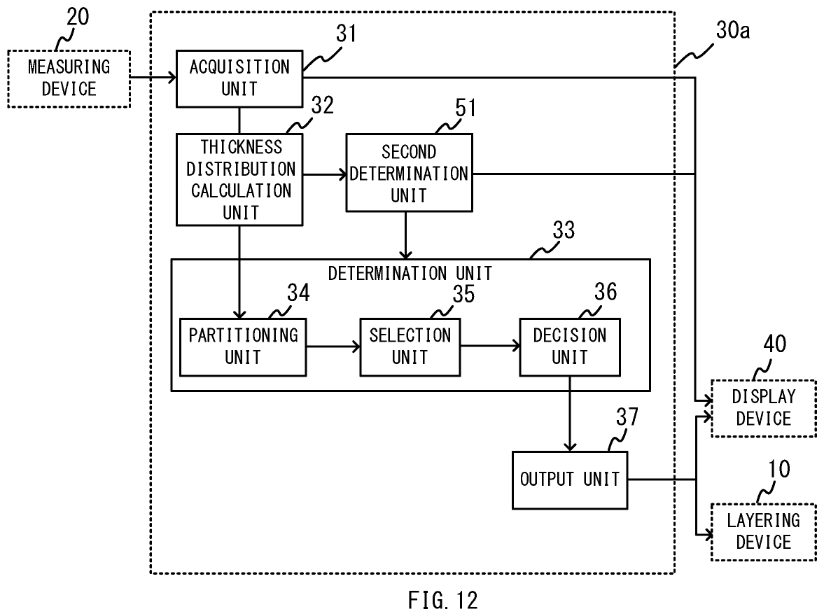

FIG. 12 is a diagram illustrating a functional configuration of a control device 30a according to the present embodiment. Referring to FIG. 12, a description will be given below of such a configuration related to a fabrication support function provided in the control device 30a according to the present embodiment. Note that the system according to the present embodiment is similar to the system 1 except for including the control device 30a in place of the control device 30.

As illustrated in FIG. 12, the control device 30a is different from the control device 30 in including a second determination unit 51. Other configurations are similar to those of the control device 30.

The second determination unit 51 determines to stop layering the new cell sheet CS on the layered cell sheet LCS on the basis of a variation in disposition of a plurality of the cell sheets CS, the variation being identified from the thickness distribution calculated by the thickness distribution calculation unit 32. In terms of meaning, the variation mentioned herein includes a degree in which the cell sheets overlap one another in the layered cell sheet LCS. A state in which the cell sheets satisfactorily overlap one another may be evaluated to have a small variation, and a state in which the cell sheets unsatisfactorily overlap one another may be evaluated to have a large variation. Specifically, the variation may be evaluated to be large, for example, when an area of a region in which layered layers having a certain thickness are formed, that is, of a region in which the cell sheets are layered on one another is smaller than a certain value. When the second determination unit 51 determines to stop the layering, for example, a result of the determination is output to the determination unit 33, and the determination processing of the layering position is stopped.

In the control device 30a configured as described above, the cell sheets CS which constitute the layered cell sheet LCS are stopped being layered when such disposition variation of these cell sheets CS exceeds a tolerance. Accordingly, automatic fabrication processing of the layered cell sheet LCS, which is poor in utilization efficiency of the cell sheets CS, can be avoided continuing.

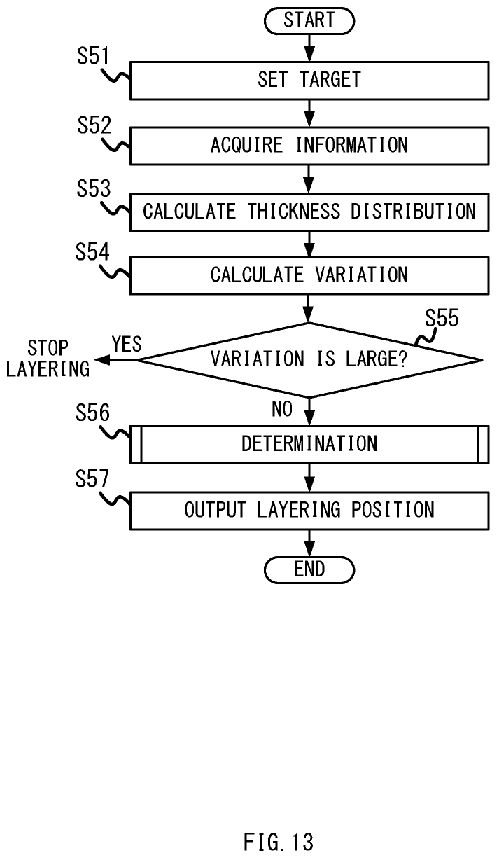

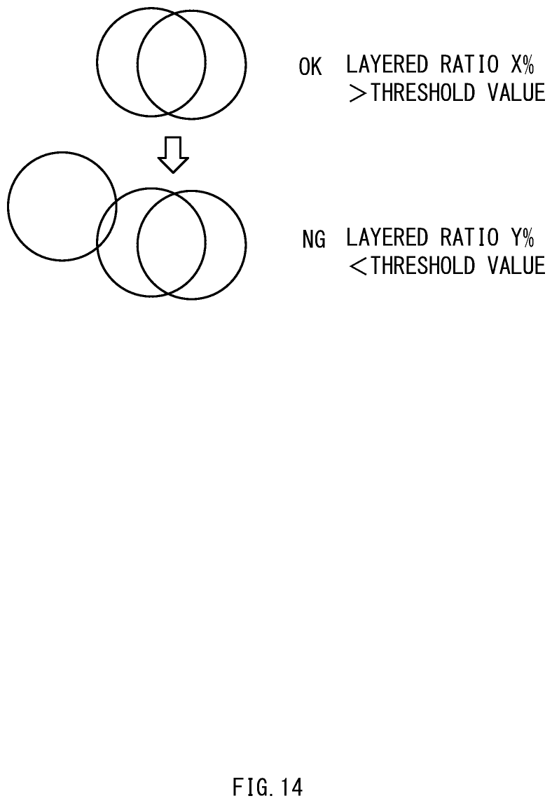

FIG. 13 is an example of a flowchart of layering support processing according to the present embodiment. FIG. 14 is a view illustrating an example of disposition of the cell sheets stopped being layered. Referring to FIGS. 13 and 14, a specific description will be given below of the layering support processing performed by the control device 30a according to the present embodiment.

When the layering support processing illustrated in FIG. 13 is started, the control device 30a first sets targets for the layered cell sheet LCS to be fabricated (step S51). Further, the acquisition unit 31 acquires an image received from the measuring device 20 (step S52), and the thickness distribution calculation unit 32 calculates the thickness distribution of the layered cell sheet LCS, which is in the acquired image, on the basis of the image (step S53). Note that the pieces of processing in steps S51 to S53 are similar to the pieces of processing in steps S11 to S13 in FIG. 4.

Thereafter, the second determination unit 51 calculates the disposition variation of the cell sheets CS (step S54). Though not particularly limited, for example, a magnitude of the disposition variation of the cell sheets CS may be evaluated on the basis of a ratio of a region where two or more cell sheets CS are mutually layered with respect to the entire region of the layered cell sheet LCS. Hereinafter, this ratio will be referred to as a layered ratio. Note that whether or not two or more cell sheets CS are mutually layered may be determined on the basis of whether or not the thickness exceeds a predetermined threshold value.

Note that the magnitude of the variation may be evaluated on the basis of an area of the region where two or more cell sheets CS are mutually layered. Hereinafter, this area will be referred to as a layered area. However, the layered area increases by repetition of the layering, and does not decrease during the automatic fabrication processing. Therefore, when a method of this evaluation is used on the basis of a constant threshold value during the automatic fabrication processing, it becomes difficult to properly evaluate the disposition variation particularly in the latter half of the automatic fabrication processing. Therefore, in the case of evaluating the variation on the basis of the layered area, for example, the threshold value which indicates a tolerance and is used in step S55 may be set for each of the number of layering times (the number of repetitions). Moreover, a reference thickness to be regarded as the layered area may be set for each of the number of layering times.

When the variation is calculated, the second determination unit 51 determines whether or not the variation is large (step S55). When the second determination unit 51 determines that the variation is large, the control device 30a stops the layering by stopping the layering support processing. Note that whether or not the variation is large enough to exceed such a tolerable range just needs to be determined by comparing the layered ratio or the layered area with such a preset threshold value, for example, as illustrated in FIG. 14.

When it is determined that the variation is not large, the determination unit 33 performs the determination processing by using the thickness distribution calculated in step S53 (step S56). Then, when the layering position is decided by the determination processing, the output unit 37 outputs the information of the layering position to the layering device 10 (step S57), and the control device 30a ends the layering support processing illustrated in FIG. 13.

Also in accordance with the control device 30a according to the present embodiment, the fabrication efficiency of the layered cell sheet can be improved as in the control device 30. Further, in the control device 30a, the automatic fabrication processing that is poor in utilization efficiency of the cell sheets CS can be prevented from being performed without restrictions. Hence, in accordance with the control device 30a, the utilization efficiency of the cell sheets CS can be improved as well as the fabrication efficiency of the layered cell sheets LCS.

(Fourth Embodiment)

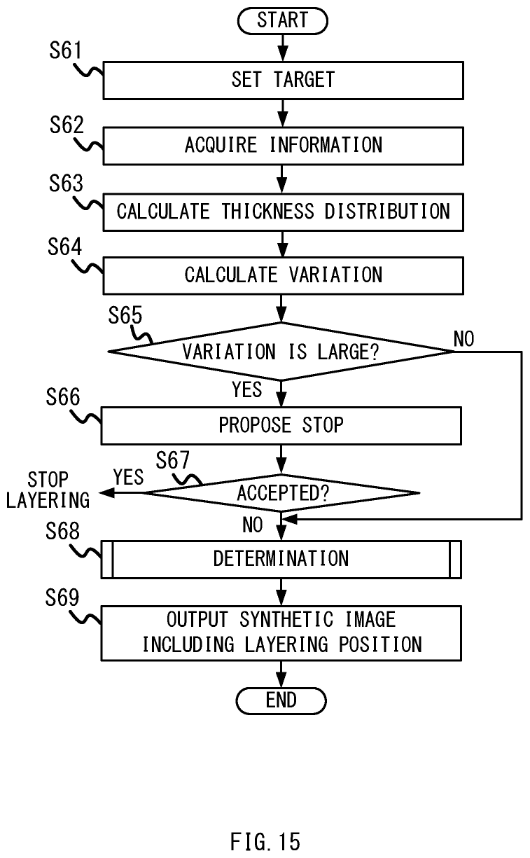

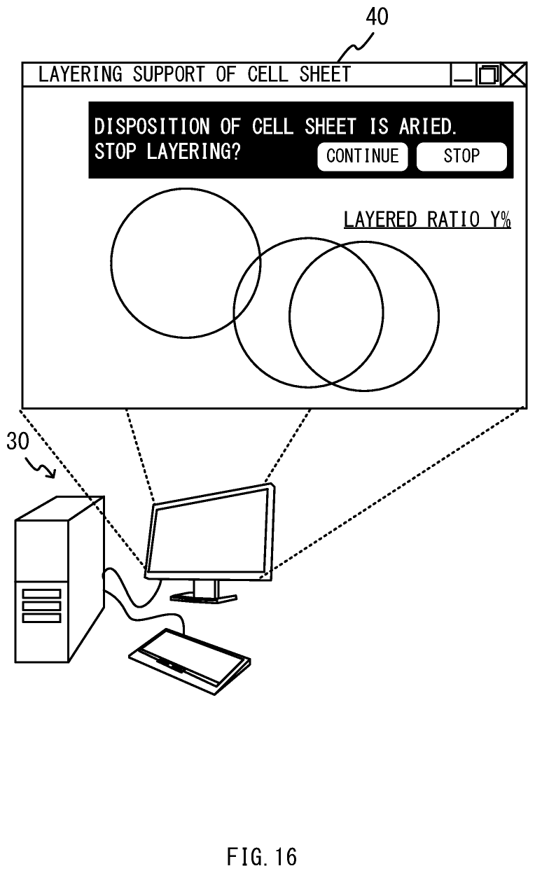

FIG. 15 is an example of a flowchart of layering support processing according to the present embodiment. FIG. 16 is a view illustrating an example of a screen to propose a stop of layering. Referring to FIGS. 15 and 16, a description will be given below of the present embodiment.

A system according to the present embodiment is different from the system according to the third embodiment in that the layering work of the cell sheet CS is manually performed. For example, in the system according to the present embodiment, the manipulator 13 of the layering device 10 is manually operated. Moreover, a control device included in the system is different from the control device 30a in outputting, to the display device 40, a synthetic image including the layering position, and in proposing the stop of the layering to the user in the case of determining to stop the layering. Note that the present embodiment is also applicable to a case where the user layers the cell sheet CS by using an instrument such as tweezers without using the layering device 10. That is, in the system according to the present embodiment, the layering device 10 may be omitted.

When the layering support processing illustrated in FIG. 15 is started, the control device first sets targets for the layered cell sheet LCS to be fabricated (step S61). Further, the acquisition unit 31 acquires an image received from the measuring device 20 (step S62), the thickness distribution calculation unit 32 calculates the thickness distribution of the layered cell sheet LCS, which is in the acquired image, on the basis of the image (step S63), and the second determination unit 51 calculates the disposition variation of the cell sheets CS (step S64), and determines whether or not the variation is large (step S65). Note that the pieces of processing in steps S61 to S65 are similar to the pieces of processing in steps S51 to S55 in FIG. 13.

In the case of determining that the variation is large, the control device proposes the stop of the layering (step S66). Herein, for example, as illustrated in FIG. 16, the display device 40 may display a screen to propose the stop of the layering on the basis of an output from the control device, and may allow the user to select whether or not to stop the layering (step S67). When the user accepts the proposal to select to stop the layering, the control device stops the layering support processing.

When the user does not select to stop the layering in step S67, or when the control device determines that the variation is not large in step S65, the determination unit 33 performs the determination processing by using the thickness distribution calculated in step S63 (step S68). Then, when the layering position is decided by the determination processing, the output unit 37 outputs a synthetic image including the layering position (step S69), and the control device ends the layering support processing illustrated in FIG. 15.

The control device outputs the synthetic image, whereby the synthetic image including the layering position is displayed on the display device 40. Therefore, the control device can support the layering work of the cell sheet CS, which is manually performed by the user by using the manipulator 13 or an instrument such as tweezers. Moreover, when the utilization efficiency of the cell sheets CS is poor, the control device can prompt the user to stop the fabrication. Hence, in accordance with the control device according to the present embodiment, the utilization efficiency of the cell sheets CS can be improved as well as the fabrication efficiency of the layered cell sheets LCS.

(Fifth Embodiment)

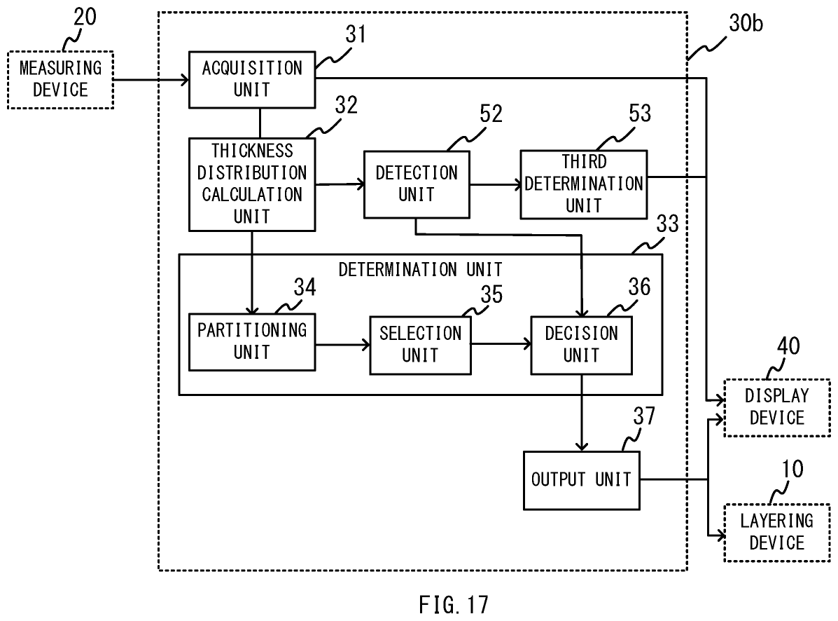

FIG. 17 is a diagram illustrating a functional configuration of a control device 30b according to the present embodiment. Referring to FIG. 17, a description will be given below of such a configuration related to a fabrication support function provided in the control device 30b according to the present embodiment. Note that a system according to the present embodiment is similar to the system 1 except for including the control device 30b in place of the control device 30.

As illustrated in FIG. 17, the control device 30b is different from the control device 30 in including a detection unit 52 and a third determination unit 53. Other configurations are similar to those of the control device 30.

The detection unit 52 detects a damaged portion in the layered cell sheet LCS on the basis of the thickness distribution calculated by the thickness distribution calculation unit 32. The damaged portion is different from a periphery thereof in thickness by at least a thickness of one cell sheet CS. Further, the damaged portion is usually local. Therefore, the damaged portion is distinguishable from such a region different in thickness from a periphery thereof, the region occurring simply due to a difference in the number of layered cell sheets CS, and is detectable on the basis of the thickness distribution. The detection unit 52 detects the damaged portion by using such a feature.

Note that the damaged portion means, for example, a portion in which a hole is made by breakage of the cell sheet CS, which occurs when the cell sheet CS is conveyed by the manipulator 13. Moreover, the damaged portion may be not only the hole but also a breach generated in the cell sheet CS. In the layered cell sheet LCS including such a damaged portion, there is a possibility that the damaged portion may expand more largely, for example, by external force generated when the layered cell sheet LCS is conveyed, and so on. Therefore, desirably, such a layered cell sheet LCS that does not include the damaged portion is fabricated.

When the detection unit 52 detects the damaged portion, a result of the detection is output to the decision unit 36 and the third determination unit 53. The decision unit 36 decides the layering position so that the damaged portion detected by the detection unit 52 and the cell sheet CS to be newly layered do not overlap each other.

The third determination unit 53 determines to stop layering the new cell sheet CS on the layered cell sheet LCS according to a position of the damaged portion detected by the detection unit 52. For example, when the damaged portion is located near the center of the layered cell sheet LCS, it becomes difficult to fabricate such a layered cell sheet LCS with a target size while going around the damaged portion. Therefore, the third determination unit 53 may determine to stop the layering, for example, on the basis of whether or not the position of the damaged portion is located near the center of the layered cell sheet LCS. When the third determination unit 53 determines to stop the layering, for example, a result of the determination is output to the determination unit 33, and the determination processing of the layering position is stopped.

In the control device 30b configured as described above, the layering is stopped on the basis of whether or not the damaged portion is present in the layered cell sheet LCS and on the basis of the position of damaged portion. Therefore, such a layered cell sheet LCS with low quality can be avoided being fabricated. Moreover, even if the layering is not stopped, the layering of the cell sheet CS is continued while going around the damaged portion. Accordingly, the damaged portion can be avoided being included in the target-size region of the layered cell sheet LCS as a final product. Hence, in accordance with the control device 30b, such a layered cell sheet LCS with high quality can be fabricated.

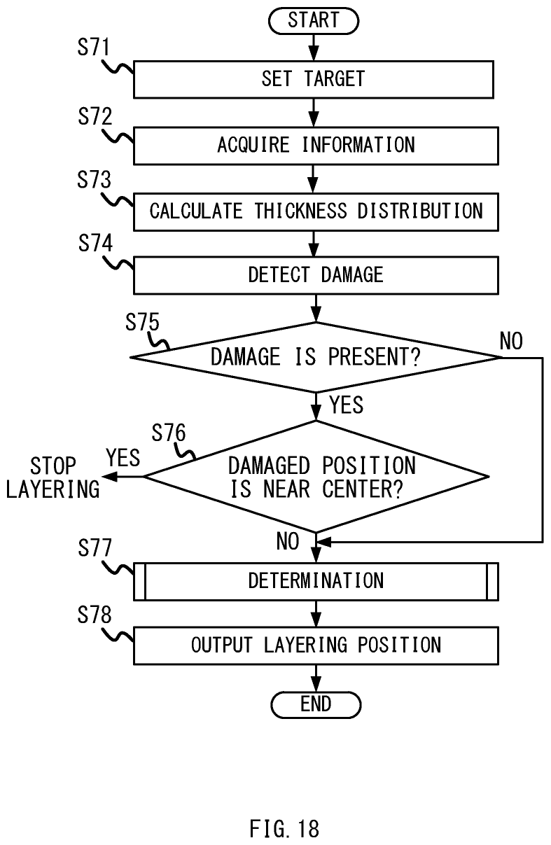

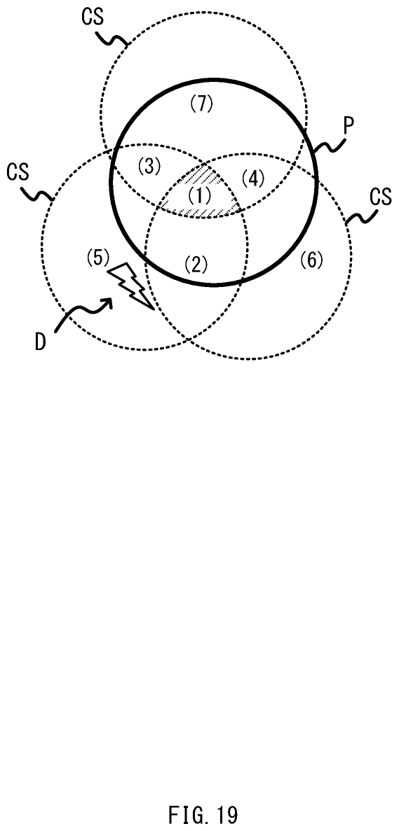

FIG. 18 is an example of a flowchart of layering support processing according to the present embodiment. FIG. 19 is a view illustrating an example of a layering position determined by determination processing according to the present embodiment. Referring to FIGS. 18 and 19, a specific description will be given below of the layering support processing performed by the control device 30b according to the present embodiment.

When the layering support processing illustrated in FIG. 18 is started, the control device 30b first sets targets for the layered cell sheet LCS to be fabricated (step S71). Further, the acquisition unit 31 acquires an image from the measuring device 20 (step S72), and the thickness distribution calculation unit 32 calculates the thickness distribution of the layered cell sheet LCS, which is in the acquired image, on the basis of the image (step S73). Note that the pieces of processing in steps S71 to S73 are similar to the pieces of processing in steps S11 to S13 in FIG. 4.

Thereafter, the detection unit 52 detects the damaged portion from the layered cell sheet LCS on the basis of the thickness distribution detected in step S73 (step S74). Then, when the damaged portion is found (step S75), the third determination unit 53 determines whether or not the position of the damaged portion is near the center of the layered cell sheet LCS (step S76). When the third determination unit 53 determines that the position is near the center, the control device 30b stops the layering support processing. Note that a criterion for determining whether or not the position of the damaged portion is near the center may be changed, for example, according to the target size. For example, when the target size is small, even if the damaged portion is relatively near the center, it is possible to fabricate the layered cell sheet LCS with the target size while going around the damaged portion. Therefore, as the target size is larger, a region corresponding to the vicinity of the center may be set wider.

When it is determined that the position of the damaged portion is not near the center, the determination unit 33 performs the determination processing by using the thickness distribution calculated in step S73 and the position of the damaged portion detected in step S74 (step S77). Thus, for example, as illustrated in FIG. 19, even if a damaged portion D is present, a layering position P can be decided while going around a damaged portion D. Then, when the layering position is decided by the determination processing, the output unit 37 outputs the information of the layering position to the layering device 10 (step S78), and the control device 30b ends the layering support processing illustrated in FIG. 18.

Also in accordance with the control device 30b according to the present embodiment, the fabrication efficiency of the layered cell sheet can be improved. Moreover, in the control device 30b, even if the damaged portion is included in the cell sheet CS for use in fabricating the layered cell sheet LCS, the damaged portion can be avoided being included in the target-size region (that is, a final product) of the layered cell sheet LCS. Hence, in accordance with the control device 30b, the fabrication of such a layered cell sheet LCS with high quality can be supported. Moreover, on the basis of the damaged position, it is determined whether or not it is appropriate to continue the layering, whereby a situation where the layering processing is redone from the start can be avoided as much as possible. Hence, even if the cell sheet is damaged, the cell sheet can be effectively utilized to be layered.

(Sixth Embodiment)

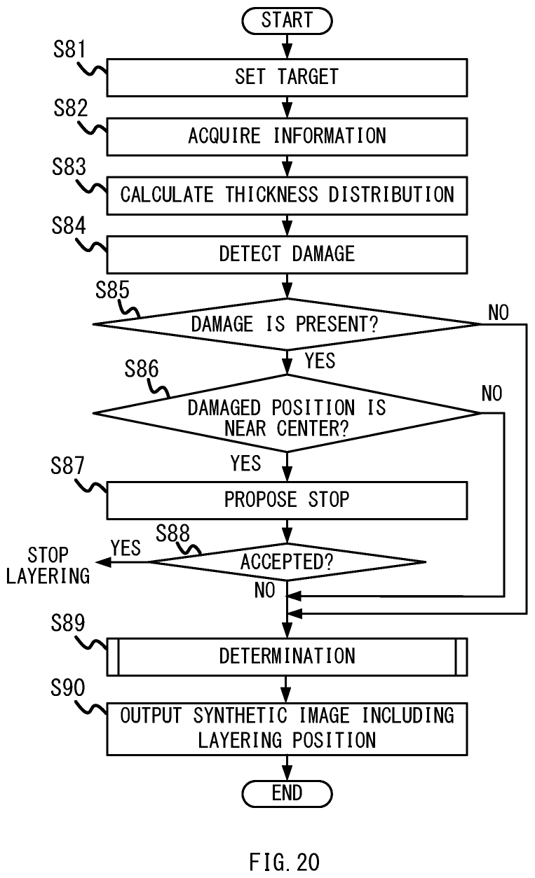

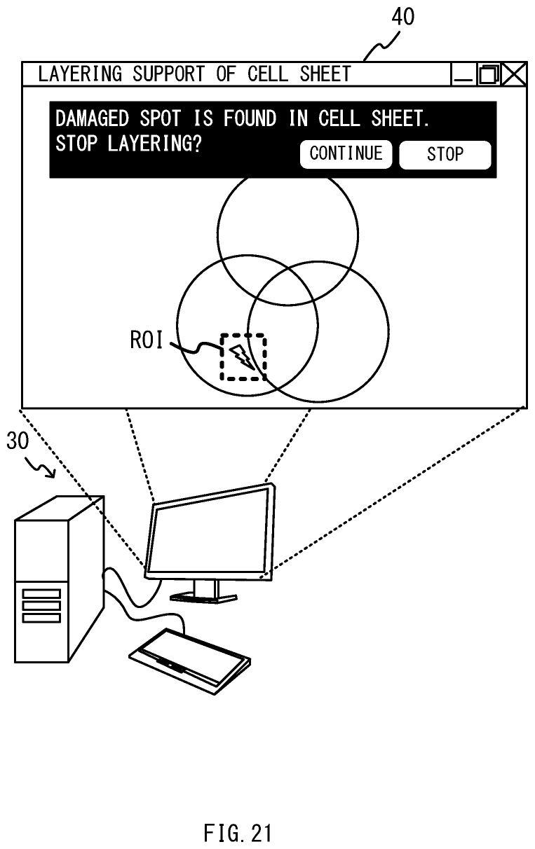

FIG. 20 is an example of a flowchart of layering support processing according to the present embodiment. FIG. 21 is a view illustrating another example of the screen to propose the stop of the layering. Referring to FIGS. 20 and 21, a description will be given below of the present embodiment.

A system according to the present embodiment is different from the system according to the fifth embodiment in that the layering work of the cell sheet CS is manually performed. For example, in the system according to the present embodiment, the manipulator 13 of the layering device 10 is manually operated. Moreover, a control device included in the system is different from the control device 30b in outputting, to the display device 40, a synthetic image including the layering position, and in proposing the stop of the layering to the user in the case of determining to stop the layering.

When the layering support processing illustrated in FIG. 20 is started, the control device first sets targets for the layered cell sheet LCS to be fabricated (step S81). Further, the acquisition unit 31 acquires an image received from the measuring device 20 (step S82), and the thickness distribution calculation unit 32 calculates the thickness distribution of the layered cell sheet LCS, which is in the acquired image, on the basis of the image (step S83). Further, the detection unit 52 detects the damaged portion from the layered cell sheet LCS on the basis of the thickness distribution detected in step S83 (step S84), and determines whether or not the damaged portion is present (step S85). When the damaged portion is found, the third determination unit 53 determines whether or not the position of the damaged portion is near the center of the layered cell sheet LCS (step S86). Note that the pieces of processing in steps S81 to S86 are similar to the pieces of processing in steps S71 to S76 in FIG. 18.

In the case of determining that the damaged portion is near the center, the control device proposes the stop of the layering (step S87). Herein, for example, as illustrated in FIG. 21, the display device 40 may display a screen to propose the stop of the layering on the basis of an output from the control device, and may allow the user to select whether or not to stop the layering (step S88). Note that, at this time, desirably, a mark ROI that indicates the damaged portion as a region of interest is displayed, whereby the user is allowed to grasp the position of the damaged portion. When the user accepts the proposal to select to stop the layering, the control device stops the layering support processing.

When the user does not select to stop the layering in step S87, or when the third determination unit 53 determines in step S86 that the damaged portion is not near the center, the determination unit 33 performs the determination processing by using the thickness distribution calculated in step S83 and the position of the damaged portion detected in step S84 (step S89). When the detection unit 52 determines in step S85 that the damaged portion is not present, the determination unit 33 performs the determination processing by using the thickness distribution calculated in step S83 (step S89). Then, when the layering position is decided by the determination processing, the output unit 37 outputs a synthetic image including the layering position (step S90), and the control device ends the layering support processing illustrated in FIG. 20.

The control device outputs the synthetic image, whereby the synthetic image including the layering position is displayed on the display device 40. Therefore, the control device can support the layering work of the cell sheet CS, which is manually performed by the user. Further, when it is difficult to fabricate the layered cell sheet LCS with the target size while going around the damaged portion, the control device can prompt the user to stop the fabrication. Hence, in accordance with the control device according to the present embodiment, such a layered cell sheet LCS with high quality can be fabricated with high fabrication efficiency. Moreover, on the basis of the damaged position, it is determined whether or not it is appropriate to continue the layering, whereby a situation where the layering processing is redone from the start can be avoided as much as possible. Hence, even if the cell sheet is damaged, the cell sheet can be effectively utilized to be layered.

(Seventh Embodiment)

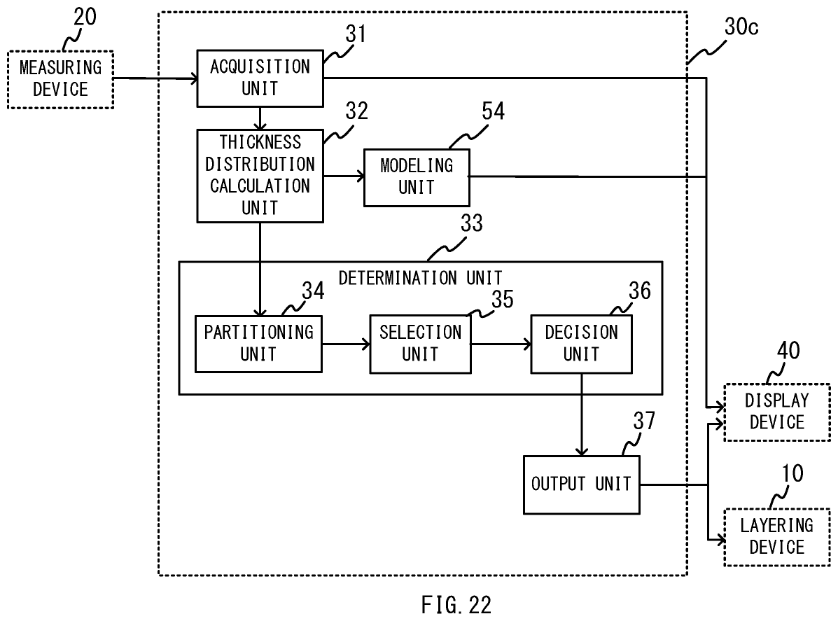

FIG. 22 is a diagram illustrating a functional configuration of a control device 30c according to the present embodiment. Referring to FIG. 22, a description will be given below of such a configuration related to a fabrication support function provided in the control device 30c according to the present embodiment. Note that a system according to the present embodiment is similar to the system 1 except for including the control device 30c in place of the control device 30.

As illustrated in FIG. 22, the control device 30c is different from the control device 30 in including a modeling unit 54. Other configurations are similar to those of the control device 30.

The modeling unit 54 generates a model image in which the thickness distribution calculated in the thickness distribution calculation unit 32 is visualized. The model image may be a heat map image that represents the thickness distribution of the layered cell sheet LCS by colors, or may be a contour line image that represents a change of the thickness of the layered cell sheet LCS by intervals of contour lines. The model image generated by the modeling unit 54 is output to the display device 40.

In the control device 30c configured as described above, the model image is output to the display device 40 in place of the information of the layering position, and accordingly, the user can grasp the thickness distribution in addition to the layering position by confirming the information displayed on the display device 40. On the basis of the layering position and the thickness distribution, the user may decide the position at which the cell sheet CS is to be actually layered.

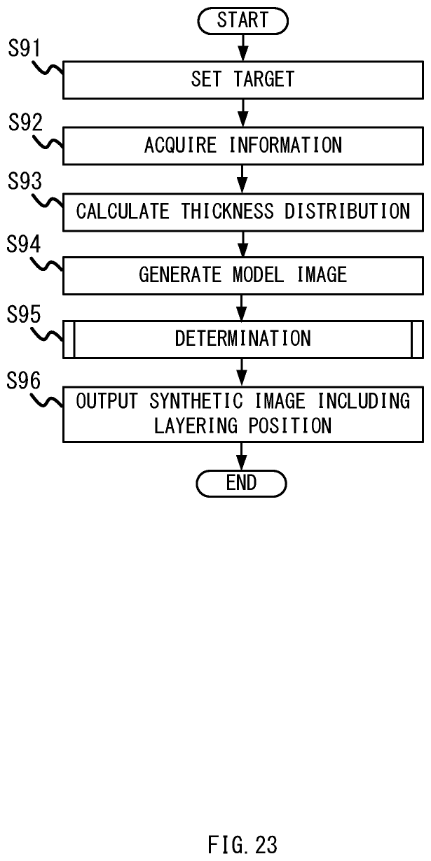

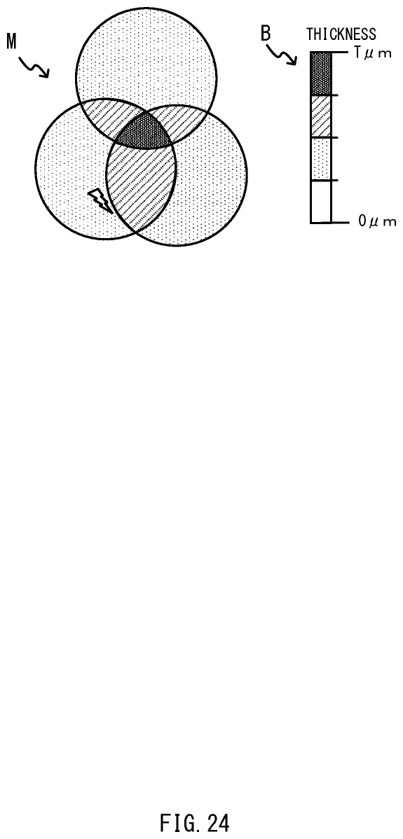

FIG. 23 is an example of a flowchart of layering support processing according to the present embodiment. FIG. 24 is a view illustrating an example of the model image. Referring to FIGS. 23 and 24, a specific description will be given below of the layering support processing performed by the control device 30c according to the present embodiment.

When the layering support processing illustrated in FIG. 23 is started, the control device 30c first sets targets for the layered cell sheet LCS to be fabricated (step S91). Further, the acquisition unit 31 acquires an image from the measuring device 20 (step S92), and the thickness distribution calculation unit 32 calculates the thickness distribution of the layered cell sheet LCS, which is in the acquired image, on the basis of the image (step S93). Note that the pieces of processing in steps S91 to S93 are similar to the pieces of processing in steps S11 to S13 in FIG. 4.

Thereafter, the modeling unit 54 generates the model image in which the thickness distribution is visualized on the basis of the thickness distribution detected in step S93 (step S94), and outputs the model image to the display device 40. Herein, for example, the modeling unit 54 may generate, as a model image M, such a heat map image as illustrated in FIG. 24. Moreover, together with the model image M, the modeling unit 54 may generate a color bar B that indicates a relationship between the thickness of the layered cell sheet LCS and colors of the model image M.

Further, the determination unit 33 performs the determination processing by using the thickness distribution calculated in step S93 (step S95). When the layering position is decided by the determination processing, the output unit 37 outputs, to the display device 40, a synthetic image including the layering position (step S96), and the control device 30c ends the layering support processing illustrated in FIG. 23. Note that, desirably, the display device 40 displays the model image output in step S94 and the synthetic image output in step S96 in association with each other, and may display these in a superimposing manner

Also in accordance with the control device 30c according to the present embodiment, the layering work of the cell sheet CS, which is manually performed by the user, can be supported by the images displayed on the display device 40. Further, the thickness distribution is visualized in addition to the layering position, whereby the user can be provided with determination criteria for determining appropriateness of the layering position recommended by the control device 30c.

(Eighth Embodiment)

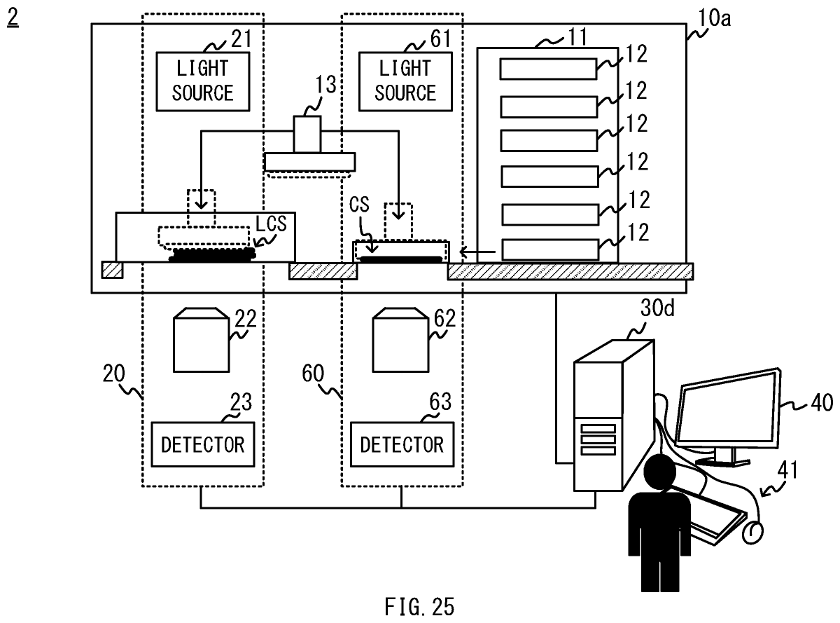

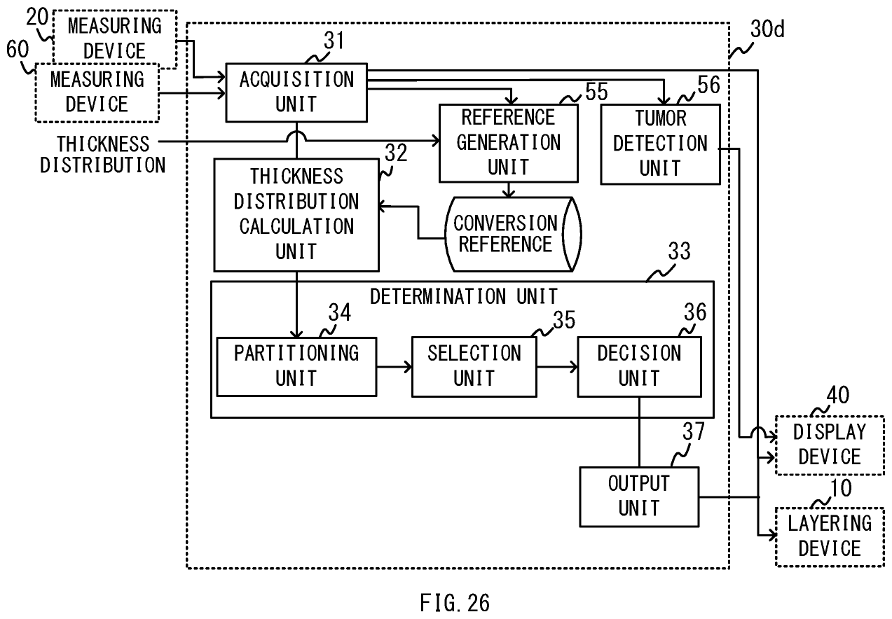

FIG. 25 is a diagram illustrating a configuration of a system 2 according to the present embodiment. FIG. 26 is a diagram illustrating a functional configuration of a control device 30d according to the present embodiment. The system 2 is similar to the system 1 in including a layering device, a measuring device, and a control device. However, the system 2 is different from the system 1 in including: a layering device 10a in place of the layering device 10; a measuring device 60 in addition to the measuring device 20; and a control device 30d in place of the control device 30.

The measuring device 60 measures the optical properties of the cell sheet CS to be newly layered on the layered cell sheet LCS. The system 2 includes the measuring device 60 in addition to the measuring device 20, whereby, in the system 2, the cell sheets CS to be layered can be inspected in advance.

The layering device 10a is different from the layering device 10 in corresponding to both of the measuring device 20 and the measuring device 60, and is similar to the layering device 10 in other points.

As illustrated in FIG. 26, the control device 30d is different from the control device 30 in including a reference generation unit 55 and a tumor detection unit 56. Other configurations are similar to those of the control device 30.

The reference generation unit 55 generates a conversion reference for converting optical information into the thickness distribution. The optical properties of the cell sheets CS each having the known thickness distribution are measured by the measuring device 60, and on the basis of information (for example, an image) regarding the optical properties acquired by the measuring device 60, the reference generation unit 55 generates such a conversion reference for converting the optical information of the layered cell sheet LCS into the thickness distribution. The thickness distribution calculation unit 32 calculates the thickness distribution of the layered cell sheet LCS by using the conversion reference generated by the reference generation unit 55.

The tumor detection unit 56 determines whether or not a tumor is present in each of the cell sheet CS. The tumor detection unit 56 may determine whether or not the tumor is present in the cell sheet CS on the basis of the image, or may determine whether or not the tumor is present in the cell sheet CS on the basis of the calculated thickness distribution of the cell sheet CS. When the tumor detection unit 56 finds a tumor, the control device 30d may stop using such a cell sheet CS from which the tumor is found. Note that, for example, the tumor is detected on the basis of irregularities identified from the thickness distribution; however, the tumor may be detected by object detection for the image of the cell sheet CS, the object detection using a machine learning model.

In the control device 30c, the image of the cell sheet CS is acquired before the layering is started, whereby the conversion reference for calculating the thickness distribution is generated. Therefore, even if a cell sheet in which a transmittance is unknown is used, the thickness distribution can be calculated by using the conversion reference generated from an image thereof. Hence, the layered cell sheet LCS can be fabricated by using an arbitrary type of cell sheet.

Further, in the control device 30c, the image of each cell sheet CS is acquired before the layering is started, whereby the cell sheets CS for use in fabricating the layered cell sheet LCS can be inspected in advance. Therefore, it is possible to fabricate the layered cell sheet LCS by using the cell sheets CS which have no tumor, and the layered cell sheet LCS with high quality can be fabricated.

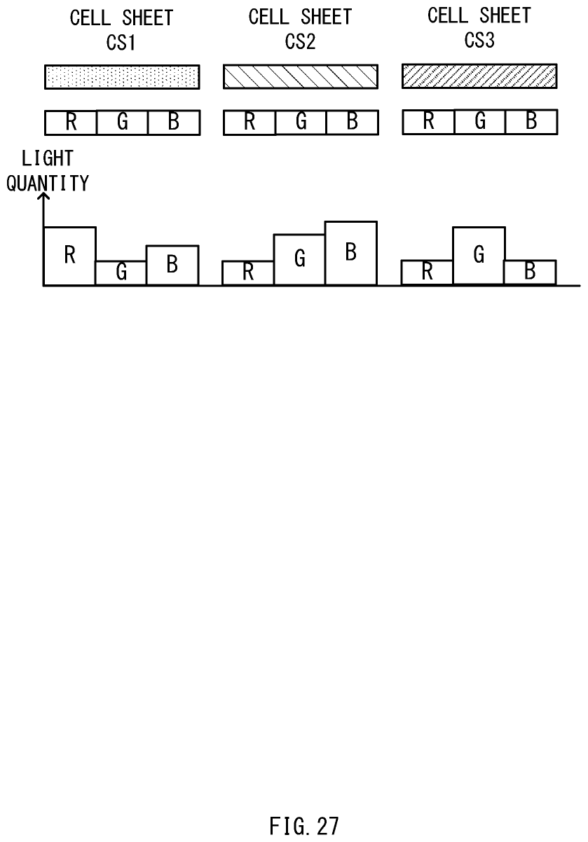

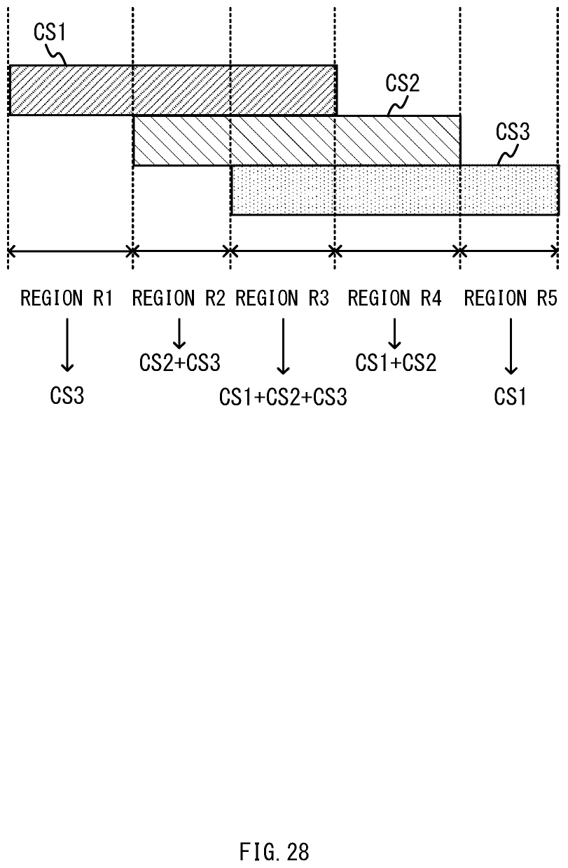

FIG. 27 is a view illustrating a relationship between types of the cell sheets and light quantities detected therefrom. FIG. 28 is a view for explaining a method of determining a layered state of different types of the cell sheets. An illustrative description has been given above of the case of handling the same type of cell sheets in which a transmittance per unit thickness is constant; however, the layered cell sheet LCS may be fabricated by using plural types of cell sheets. Referring to FIGS. 27 and 28, a description will be given below of a method for supporting the fabrication of the layered cell sheet LCS by using the plural types of cell sheets.

The different types of cell sheets may sometimes be different from one another in transmittance per unit thickness, and it is difficult to identify the thickness of each thereof from a light quantity. In such a case, the thickness just needs to be identified by using light with a plurality of wavelengths. As illustrated in FIG. 27, for each of plural types of the cell sheets (a cell sheet CS1, a cell sheet CS2, a cell sheet CS3) for use in fabricating the layered cell sheet LCS, a transmittance thereof is measured by the plurality of wavelengths to grasp a balance thereof in advance. In this way, even if the plural types of cell sheets are used for the layering, it is possible to identify the types of cell sheets and a combination thereof as illustrated in FIG. 28, and the thickness distribution can be calculated accurately.

The combination of the plurality of cell sheets is identified, whereby a layered cell sheet that is more complicated can be fabricated by layering the plural types of cell sheets. Moreover, it can also be inspected whether or not the layered cell sheet is fabricated by a proper combination, and therefore, the control device can be utilized not only as the fabrication support apparatus that supports the fabrication of the layered cell sheet but also as an inspection apparatus of the layered cell sheet.

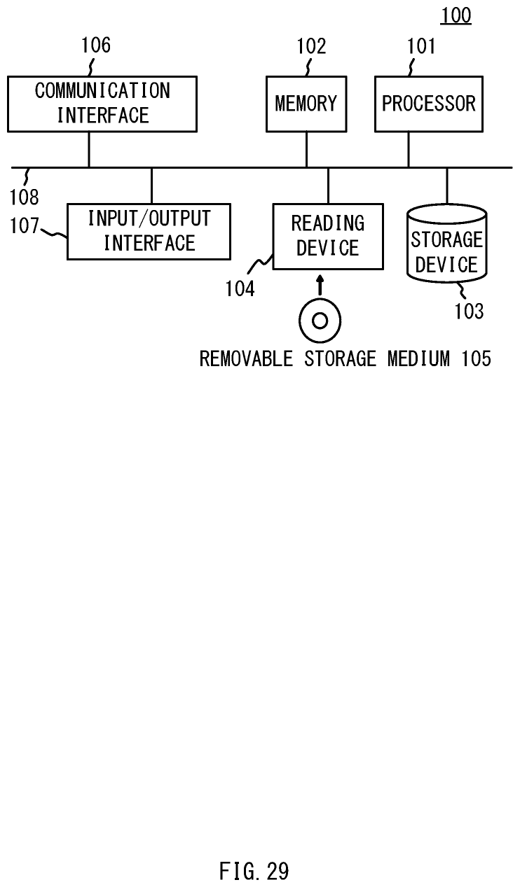

FIG. 29 is a diagram illustrating a hardware configuration of a computer 100 for achieving the control device according to the above-mentioned embodiments. As illustrated in FIG. 29, the computer 100 includes, as the hardware configuration, a processor 101, a memory 102, a storage device 103, a reading device 104, a communication interface 106, and an input/output interface 107. Note that the processor 101, the memory 102, the storage device 103, the reading device 104, the communication interface 106, and the input/output interface 107 are connected to one another, for example, via a bus 108.

For example, the processor 101 may be a single processor, a multiprocessor, or a multicore processor. The processor 101 reads out and executes a program stored in the storage device 103, and thereby operates as a variety of functional units included in the above-mentioned control device, which are the acquisition unit 31, the thickness distribution calculation unit 32, the determination unit 33, the output unit 37, the second determination unit 51, the detection unit 52, the third determination unit 53, the modeling unit 54, the reference generation unit 55, and the tumor detection unit 56.

For example, the memory 102 is a semiconductor memory, and may include a RAM region and a ROM region. For example, the storage device 103 is a hard disk, a semiconductor memory such as a flash memory, or an external storage device.

For example, the reading device 104 accesses a removable recording medium 105 in accordance with an instruction of the processor 101. For example, the removable recording medium 105 is achieved by a semiconductor device, a medium to/from which information is input/output by a magnetic action, a medium to/from which information is input/output by an optical action. Note that, for example, the semiconductor device is a universal serial bus (USB) memory. Further, the medium from/to which information is input/output by the magnetic action is, for example, a magnetic disk. The medium from/to which information is input/output by the optical action is, for example, a compact disc (CD)-ROM, a digital versatile disk (DVD), or a Blu-ray disc (Blu-ray is a registered trademark).

The communication interface 106 communicates with other devices such as the layering device 10 and the measuring device 20, for example, in accordance with an instruction of the processor 101. The input/output interface 107 is an interface between an input device/an output device and computer 100. For example, the input device is the input device 41, and is a device such as a keyboard, a mouse and a touch panel, which receives an instruction from the user. For example, the output device is the display device 40, and an audio device such as a speaker.

The above-mentioned acquisition unit 31 and output unit 37 may include at least one of the input/output interface 107 and the communication interface 106.

The program to be executed by the processor 101 is provided to the computer 100, for example, in the following forms.

(1) Installed in the storage device 103 in advance.

(2) Provided by the removable recording medium 105.

(3) Provided from a server such as a program server.

Note that the hardware configuration of the computer 100 for achieving the control device, the computer 100 being mentioned with reference to FIG. 29, is an example, and the embodiments are not limited to this. For example, the above-mentioned configuration may be partially deleted, or a new constituent may be added thereto. Moreover, in another embodiment, for example, a part or all of the functions of the above-mentioned functional units may be implemented as hardware by a field programmable gate array (FPGA), a system-on-a-chip (SoC), an application specific integrated circuit (ASIC), a programmable logic device (PLD), and the like. That is, an arbitrary electric circuit included in the control device may perform a part or all of the above-mentioned fabrication support processing, as long as the circuitry included in the control device performs the above manufacturing support process.

The above-mentioned embodiments illustrate specific examples in order to facilitate the understanding of the invention, and the present invention is not limited to these embodiments. Variations obtained by modifying the above-mentioned embodiments and alternatives to the above-mentioned embodiments can be included. That is, in the respective embodiments, the constituents can be modified without departing from the spirit and scope thereof. In addition, a new embodiment can be implemented by appropriately combining a plurality of the constituents disclosed in one or more embodiments. Further, some constituents may be deleted from the constituents illustrated in the respective embodiments, or some constituents may be added to the constituents illustrated in the embodiments. Furthermore, the processing procedures described in the respective embodiments may be performed in a different order as long as there is no contradiction. That is, the fabrication support apparatus of the layered cell sheet, the fabrication support system, the fabrication support method and the program in the present invention can be modified and altered in various ways within the scope without departing from the description of the scope of claims.

What is claimed is:

1. A fabrication support apparatus of a layered cell sheet in which a plurality of cell sheets are layered on one another, the apparatus comprising circuitry,
wherein the circuitry is configured:
to acquire optical information that is information regarding optical properties of the layered cell sheet;
to calculate a thickness distribution of the layered cell sheet based on the acquired optical information;
to determine a layering position of a cell sheet to be newly layered on the layered cell sheet based on the calculated thickness distribution; and
to output information of the determined layering position.

2. The fabrication support apparatus according to claim 1, wherein to determine a layering position of the cell sheet includes to decide the layering position so that a thickest region of the layered cell sheet and the cell sheet to be newly layered overlap each other based on the calculated thickness distribution.

3. The fabrication support apparatus according to claim 1, wherein
to determine the layering position of the cell sheet includes:
to decide the layering position so that a thickest region of the layered cell sheet and the cell sheet to be newly layered overlap each other based on the calculated thickness distribution when a region that has reached a target thickness of the layered cell sheet is not present in the layered cell sheet; and
to decide the layering position so that a region that has not reached the target thickness, the region being adjacent to the region that has reached the target thickness, and the cell sheet to be newly layered overlap each other based on the calculated thickness distribution when the region that has reached the target thickness is present in the layered cell sheet.

4. The fabrication support apparatus according to claim 2, wherein
to determine a layering position of the cell sheet includes:
to partition a region of the layered cell sheet into one or more partial regions based on the calculated thickness distribution;
to select a target partial region taken as a layering target from among the one or more partial regions based on at least one of sizes and thicknesses of the one or more partial regions; and
to decide the layering position so that the target partial region and the cell sheet to be newly layered overlap each other.

5. The fabrication support apparatus according to claim 3, wherein
to determine a layering position of the cell sheet includes:
to partition a region of the layered cell sheet into one or more partial regions based on the calculated thickness distribution;
to select a target partial region taken as a layering target from among the one or more partial regions based on at least one of sizes and thicknesses of the one or more partial regions; and
to decide the layering position so that the target partial region and the cell sheet to be newly layered overlap each other.

6. The fabrication support apparatus according to claim 1, wherein the circuitry is further configured to determine to stop layering the new cell sheet on the layered cell sheet based on a variation in disposition of the plurality of cell sheets, the variation being identified from the calculated thickness distribution.

7. The fabrication support apparatus according to claim 2, wherein the circuitry is further configured to determine to stop layering the new cell sheet on the layered cell sheet based on a variation in disposition of the plurality of cell sheets, the variation being identified from the calculated thickness distribution.

8. The fabrication support apparatus according to claim 3, wherein the circuitry is further configured to determine to stop layering the new cell sheet on the layered cell sheet based on a variation in disposition of the plurality of cell sheets, the variation being identified from the calculated thickness distribution.

9. The fabrication support apparatus according to claim 4, wherein the circuitry is further configured to determine to stop layering the new cell sheet on the layered cell sheet based on a variation in disposition of the plurality of cell sheets, the variation being identified from the calculated thickness distribution.

10. The fabrication support apparatus according to claim 5, wherein the circuitry is further configured to determine to stop layering the new cell sheet on the layered cell sheet based on a variation in disposition of the plurality of cell sheets, the variation being identified from the calculated thickness distribution.

11. The fabrication support apparatus according to claim 1, wherein the circuitry is further configured to detect a damaged portion in the layered cell sheet based on the calculated thickness distribution.

12. The fabrication support apparatus according to claim 11, wherein the circuitry is further configured to determine to stop layering the new cell sheet on the layered cell sheet according to a position of the detected damaged portion.

13. The fabrication support apparatus according to claim 1, wherein
the circuitry is further configured to generate a conversion reference for converting the optical information into the thickness distribution based on information regarding optical properties of a cell sheet having a known thickness distribution, and
to calculate a thickness distribution of the layered cell sheet includes to calculate a thickness distribution of the layered cell sheet by using the conversion reference.

14. A fabrication support system comprising:
the fabrication support apparatus according to claim 1; and
a layering device that layers a new cell sheet on the layered cell sheet based on the information of the layering position, the information being output by the fabrication support apparatus.

15. A fabrication support system comprising:
the fabrication support apparatus according to claim 1; and
a display device that displays the information of the layering position, the information being output by the fabrication support apparatus.

16. The fabrication support system according to claim 15, wherein
the optical information includes an image of the layered cell sheet, and
the display device displays the information of the layering position on the image in a superimposing manner

17. The fabrication support system according to claim 15, wherein
the circuitry is further configured to generate a model image in which the calculated thickness distribution is visualized, and
the display device displays the model image.

18. A fabrication support method of a layered cell sheet in which a plurality of cell sheets are layered on one another, the method comprising:
acquiring optical information that is information regarding optical properties of the layered cell sheet;
calculating a thickness distribution of the layered cell sheet based on the acquired optical information;
determining a layering position of a cell sheet to be newly layered on the layered cell sheet based on the calculated thickness distribution; and
outputting information of the determined layering position.

19. A non-transitory computer-readable medium that stores a program for causing a computer of a fabrication support apparatus of a layered cell sheet in which a plurality of cell sheets are layered on one another to execute processing:
for acquiring optical information that is information regarding optical properties of the layered cell sheet;
for calculating a thickness distribution of the layered cell sheet based on the acquired optical information;
for determining a layering position of a cell sheet to be newly layered on the layered cell sheet based on the calculated thickness distribution; and
for outputting information of the determined layering position.
